(19) United States
(12) Patent Application Publication
(10) Pub. No.: US 2024/0225885 A1
(43) Pub. Date: Jul. 11, 2024

(54) FLUID COLLECTION ASSEMBLIES INCLUDING A POROUS MATERIAL HAVING A FIRST POROUS LAYER, A SECOND POROUS LAYER, AND A SUPPORTING LAYER

(71) Applicant: PUREWICK CORPORATION, Covington, GA (US)

(72) Inventors: Zhihui Yin, Lilburn, GA (US); Kamil Szymaniak, Dacula, GA (US); Kathleen Davis, Atlanta, GA (US); Michael Anderson, Monroe, GA (US); Brendan Tan-Fahed, Potomac, MD (US)

(21) Appl. No.: 18/610,523

(22) Filed: Mar. 20, 2024

Related U.S. Application Data
(63) Continuation of application No. PCT/US2022/044107, filed on Sep. 20, 2022.
(60) Provisional application No. 63/247,491, filed on Sep. 23, 2021.

(51) Int. Cl. A61F 5/44; A61F 5/455; A61F 13/84

(52) U.S. Cl. CPC A61F 5/4401; A61F 5/455; A61F 2013/8494

(57) ABSTRACT

Embodiments are directed to fluid collection assemblies including a porous material having a first porous layer, a second porous layer, and a supporting layer extending between the first porous layer and the second porous layer. Embodiments are also directed towards fluid collection systems including such fluid collection assemblies and methods of forming and using such fluid collection assemblies. In an embodiment, a fluid collection assembly is disclosed. The fluid collection assembly includes a fluid impermeable layer at least defining a chamber, at least one opening, and a chamber. The fluid collection assembly also includes a porous material disposed in the chamber. The porous material includes a first porous layer, a second porous layer, and a supporting layer positioned and extending between the first porous layer and the second porous layer.

### CROSS-REFERENCE TO RELATED APPLICATION(S)

This application is a continuation of PCT International Application No. PCT/US2022/044107 filed on Sep. 20, 2022, which claims priority to U.S. Provisional Patent Application No. 63/247,491 filed on Sep. 23, 2021, the disclosure of each of which is incorporated herein, in its entirety, by this reference.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies including a porous material having a first porous layer, a second porous layer, and a supporting layer extending between the first porous layer and the second porous layer. Embodiments are also directed towards fluid collection systems including such fluid collection assemblies and methods of forming and using such fluid collection assemblies. In an embodiment, a fluid collection assembly is disclosed. The fluid collection assembly includes a fluid impermeable layer at least defining a chamber, at least one opening, and a chamber. The fluid collection assembly also includes a porous material disposed in the chamber. The porous material includes a first porous layer, a second porous layer, and a supporting layer positioned and extending between the first porous layer and the second porous layer.

In an embodiment, a fluid collection system is disclosed. The fluid collection system includes a fluid collection assembly. The fluid collection assembly includes a fluid impermeable layer at least defining a chamber, at least one opening, and a chamber. The fluid collection assembly also includes a porous material disposed in the chamber. The porous material includes a first porous layer, a second porous layer, and a supporting layer positioned and extending between the first porous layer and the second porous layer. The fluid collection system also includes a fluid storage container and a vacuum source. The chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a cross-sectional schematic of a porous material, according to an embodiment, that may be used in any of the fluid collection assemblies disclosed herein.

FIG. 2 is a cross-sectional schematic of a porous material, according to an embodiment, that may be used in any of the fluid collection assemblies disclosed herein.

FIG. 3A is an isometric view of a fluid collection assembly including a porous material, according to an embodiment.

FIGS. 3B and 3C a cross-sectional schematics of the fluid collection assembly taken along planes 3B-3B and 3C-3C, respectively, shown in FIG. 3A.

FIG. 4 is a cross-sectional schematic of a fluid collection assembly, according to an embodiment.

FIG. 5A is a cross-sectional view of a fluid collection assembly including a shapeable conduit, according to an embodiment.

FIG. 5B is a cross-sectional view of the fluid collection assembly taken along plane 5B-5B, according to an embodiment.

FIG. 6 is a cross-sectional view of a fluid collection assembly, according to an embodiment.

FIG. 7 is a cross-sectional view of a fluid collection assembly, according to an embodiment.

FIG. 8 is a block diagram of a fluid collection system for fluid collection assembly, according to an embodiment.

### DETAILED DESCRIPTION

Embodiments are directed to fluid collection assemblies including a porous material having a first porous layer, a second porous layer, and a supporting layer extending between the first porous layer and the second porous layer. Embodiments are also directed towards fluid collection systems including such fluid collection assemblies and methods of forming and using such fluid collection assemblies. An example fluid collection assembly includes a fluid impermeable layer (e.g., fluid impermeable barrier) at least defining a chamber, at least one opening, and a fluid outlet. The fluid collection assembly also includes a porous material disposed in the chamber. The porous material includes a first porous layer (e.g., fluid permeable membrane), a second porous layer, and a supporting layer. The first porous layer may be positioned in the chamber to receive bodily fluids before the second porous layer. The supporting layer is arranged to be positioned and extend between the first porous layer and the second porous layer.

During use, the fluid collection assembly may be positioned on an individual such that the porous material is positioned adjacent to a urethral opening (e.g., vaginal) or receives a urethral opening (e.g., penis). The individual may discharge one or more bodily fluids (e.g., urine, sweat, blood, etc.). The discharged bodily fluids may be received into the porous material. The bodily fluids may flow through the porous material to an inlet of a conduit positioned through a fluid outlet defined by the fluid impermeable layer. The bodily fluids then may flow through the conduit to be removed from the fluid collection assembly. In an embodiment, a vacuum may be provided from the conduit to the porous material. The vacuum may facilitate flow of the bodily fluids through the porous material to the inlet of the conduit. The vacuum may also facilitate flowing the bodily fluids through the conduit. The vacuum may be provided from a vacuum source that is in fluid communication with the conduit.

The porous materials of some conventional fluid collection assemblies include a gauze, cross-lapped porous nonwoven materials, or other porous materials that are positioned to initially receive bodily fluids from the individual using such conventional fluid collection assemblies. Such porous materials are configured to be hydrophobic or otherwise wick bodily fluids into the conventional fluid collection assemblies. However, it has been found that many of the gauzes, cross-lapped nonwoven materials, and other porous materials positioned to initially receive the bodily fluids from the individual may be inefficient at capturing the bodily fluids discharged from the individual which increases the likelihood that bodily fluids leak from the fluid collection assemblies. Further, it has been found that many of the gauzes and other porous materials remain wet after the individual discharges bodily fluids which prevents the conventional fluid collection assemblies from being used for a prolonged period of time (e.g., periods of time greater than 12 hours) without causing skin degradation of the individual.

The porous materials of the fluid collection assemblies disclosed herein (i.e., the porous materials that include a first porous layer, a second porous layer, and a supporting layer therebetween) remedy at least some of these issues associated with the porous materials of conventional fluid collection assemblies. For example, the first porous layer is configured to efficiently receive the bodily fluids, thereby preventing or at least inhibiting leakage of the bodily fluids. The first porous layer may also be configured to dry relatively quickly after receiving the bodily fluids which allows the fluid collection assemblies disclosed herein to be used for prolonged periods of time (e.g., periods of time greater than about 24 hours, such as about 24 hours to about 36 hours, about 30 hours to about 42 hours, or about 36 hours to about 48 hours). The first porous layer may efficiently receive the bodily fluids and remain dry, for example, due to at least one or more of the hydrophilicity of the first porous layer, the average macroscopic pore size of the first porous layer, the thickness of the first porous layer, or the properties of the other layers of the porous material, as will be discussed in more detail below. The second porous layer promotes flow of the bodily fluids out of the porous material and towards an inlet of a conduit which may remove the bodily fluids from the fluid collection assembly. The second porous layer promotes such fluid flow, for example, due to at least one or more of the hydrophobicity of the second porous layer, the average macroscopic pore size of the second porous layer, or the properties of the other materials of the porous material, as discussed in more detail below. The supporting layer maintains the distance between the first and second porous layers and provides an effective pathway for bodily fluids to flow therethrough. In particular, the supporting layer provides an effective pathway for bodily fluids to flow towards the inlet of the conduit. The supporting layer may provide the effective pathway due to, for example, at least one or more of the relatively high percent void space thereof, the fibers forming the supporting layer, the direction that the fibers extends, or the properties of the other layers of the porous material.

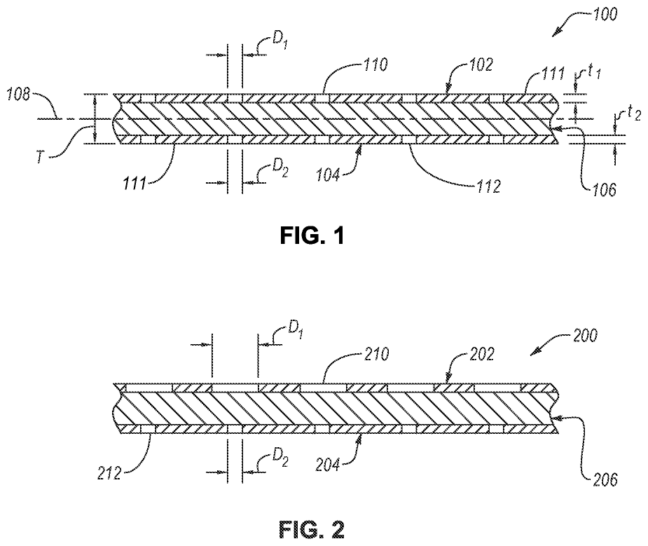

FIG. 1 is a cross-sectional schematic of a porous material 100, according to an embodiment, that may be used in any of the fluid collection assemblies disclosed herein. The porous material 100 includes a first porous layer 102, a second porous layer 104 on a second side of the porous material 100, and a supporting layer 106 extending between the first and second porous layers 102, 104. Generally, the porous material 100 is positioned to receive bodily fluids discharged from the urethral opening of an individual before the second porous layer 104 and the supporting layer 106. As such, during use, the first porous layer 102 may be positioned closer to the opening or otherwise positioned closer to the urethral opening than a corresponding (e.g., adjacent) portion of the second porous layer 104 and the supporting layer 106.

As previously discussed, the first porous layer 102 is configured to receive the bodily fluids discharged by the urethral opening of the individual before the second porous layer 104 and the supporting layer 106. As such, the first porous layer 102 is configured to efficiently receive bodily fluids to prevent or at least inhibit bodily fluids leaking from the porous material 100. As used herein, efficiently receive bodily fluids refers to the ability of the first porous layer 102 to receive and have flow therethrough a large volume of bodily fluids over a short period of time. For example, the first porous layer 102 may efficiently receive the bodily fluids when the first porous layer 102 may receive and/or have flow therethrough about 6 ml/s or more of bodily fluids, such as about 10 ml/s or more, about 20 ml/s or more, about 30 ml/s or more, about 40 ml/s or more, about 50 ml/s or more, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s.

Also, due the proximity of the first porous layer 102 to the urethral opening of the individual, the first porous layer 102 is configured to dry relatively quickly after receiving the bodily fluids thereby preventing or at least inhibiting skin degradation when the fluid collection assembly including the porous material 100 is used for prolonged periods of time. As used herein, the first porous layer 102 dries relatively quickly when the bodily fluids form about 10 wt % or less (e.g., about 7.5 wt % or less, about 5 wt % or less, about 2.5 wt % or less, or about 1 wt % or less) of the first porous layer 102 about 1 hour or less (e.g., about 45 minutes or less, about 30 minutes or less, about 15 minutes or less, about 10 minutes or less, about 5 minutes or less, or about 1 minute or less) after the first porous layer 102 receives the bodily fluids.

In an example, the first porous layer 102 may efficiently receive the bodily fluids because the first porous layer 102 is hydrophilic. When the first porous layer 102 is hydrophilic, the first porous layer 102 pulls the bodily fluids into the first porous layer 102, thereby allowing the first porous layer 102 to efficiently receive the bodily fluids. Also, the hydrophilic first porous layer 102 distributes the bodily fluids through the first porous layer 102, which allows the first porous layer 102 to receive a large quantity of bodily fluids and facilitate transferring the bodily fluids from the first porous layer 102 to the supporting layer 106. The first porous layer 102 may be hydrophilic when the first porous layer 102 exhibits a contact angle with water that is 90° or less, such as about 80° or less, about 70° or less, about 60° or less, about 50° or less, about 40° or less, about 30° of less, about 20° or less, about 10° or less, or in ranges of about 0° to about 20°, about 10° to about 30°, about 20° to about 40°, about 30° to about 50°, about 40° to about 60°, about 50° to about 70°, about 60° to about 80°, or about 70° to 90°. Generally, increasing the hydrophilicity (i.e., decreasing the contact angle with water) of the first porous layer 102 improves the efficiency at which the first porous layer 102 may receive bodily fluids. However, increasing the hydrophilicity of the first porous layer 102 may make drying the first porous layer 102 quickly difficult. As such, the hydrophilicity of the first porous layer 102 may be selected based on balancing these two factors, based on which need (efficiently receiving the bodily fluids or quickly drying the first porous layer 102) is more important in a particular application. In an embodiment, the first porous layer 102 is formed from a hydrophilic material. In an embodiment, the first porous layer 102 is formed from a material (e.g., a hydrophobic material) that is treated to increase a hydrophilicity thereof or a base layer that is coated with a hydrophilic material.

The first porous layer 102 may exhibit a thickness t₁ that is significantly less than a thickness of top layers conventionally used in the porous materials of conventional fluid collection assemblies (e.g., the thickness of the top layers of conventional fluid collection assemblies may be greater than 1 mm). For example, the thickness t₁ of the first porous layer 102 may be about 500 μm or less, such as about 400 μm or less, about 300 μm or less, about 250 μm or less, about 200 μm or less, about 150 μm or less, about 100 μm or less, or in ranges of about 50 μm to about 150 μm, about 100 μm to about 200 μm, about 150 μm to about 250 μm, about 200 μm to about 300 μm, about 250 μm to about 400 μm, or about 300 μm to about 500 μm. The thickness t₁ of the first porous layer 102 may allow the first porous layer 102 to efficiently receive the bodily fluids since the distance that the bodily fluids need to flow through the first porous layer 102 is decreased. The thickness t₁ of the first porous layer 102 may also allow the first porous layer 102 to quickly dry since the thickness t₁ of the first porous layer 102 only allows the first porous layer 102 to hold a relatively small quantity of bodily fluids at any given time. The relatively small quantity of bodily fluids present in the first porous layer 102 may be easily removed (e.g., evaporated) into the atmosphere or by air flow caused by a vacuum applied to the chamber of the fluid collection assembly. The limited quantity of bodily fluids held in the first porous layer 102 may allow the first porous layer 102 to be formed from a hydrophilic material. For example, conventional selection of materials for fluid collection assemblies avoids using hydrophilic materials, especially in portions proximate to the urethral opening, since hydrophilic materials tend to retain the bodily fluids and remain wet. As such, conventional selection of materials for fluid collection assemblies tend to use hydrophobic materials (i.e., materials exhibiting a contact angle with water that is greater than 90°) since hydrophobic materials do not retain large quantities of fluid. However, hydrophobic materials may not effectively receive bodily fluids.

It is noted that, generally, decreasing the thickness t₁ increases the efficiency at which the first porous layer 102 receives the bodily fluids and increases how quickly the first porous layer 102 may dry. However, decreasing the thickness t₁ decreases the durability of the first porous layer 102. Decreasing the thickness t₁ may also limit diffusion of the bodily fluids into the first porous layer 102 in a direction that is generally parallel to a longitudinal axis 108 which, in turn, may facilitate flow of the bodily fluids from the first porous layer 102 into the supporting layer 106.

The first porous layer 102 may define a plurality of first macroscopic pores 110 extending at least partially therethrough. The first macroscopic pores 110 include pores exhibiting a maximum dimension measured perpendicular to the longitudinal axis 108 that is about 1 mm or greater. The first macroscopic pores 110 may exhibit a first average macroscopic pore size D₁, measured perpendicularly to the longitudinal axis 108, that is about 1 mm or more, about 2 mm or more, about 3 mm or more, about 4 mm or more, about 5 mm or more, about 6 mm or more, about 7 mm or more, about 8 mm or more, about 9 mm or more, about 10 mm or more, or in ranges of about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, or about 8 mm to about 10 mm. The first average macroscopic pore size D₁ may be determined using the maximum dimension of the first macroscopic pores 110 or the mean dimension of the first macroscopic pores 110. Generally, increasing the first average macroscopic pore size D₁ increases how efficiently the first porous layer 102 receives the bodily fluids. However, increasing the first average macroscopic pore size D₁ increases the surface roughness of the first porous layer 102 which, in turn, may make the fluid collection assembly less comfortable to use. It is noted that the first porous layer 102 may also define a plurality of microscopic pores (not shown). The microscopic pores include pores exhibiting a maximum dimension, measured perpendicularly to the longitudinal axis 108, which is about 1 mm or less.

The first macroscopic pores 110 may exhibit any suitable cross-sectional shape taken along a plane that is parallel to the longitudinal axis 108 and to an outer surface 111 of the first porous layer 102. For example, the first macroscopic pores 110 may exhibit a generally circular cross-sectional shape, generally rectangular (e.g., square) cross-sectional shape, a generally pentagonal cross-sectional shape, a generally hexagonal cross-sectional shape, a generally octagonal cross-sectional shape, a generally oval or ellipsoidal cross-sectional shape, a generally oblong cross-sectional shape, or any other suitable shape. The cross-sectional shape of the first macroscopic pores 110 may affect how efficiently the first porous layer receives the bodily fluids and how quickly the first porous layer 102 dries. For example, cross-sectional shapes that cause the first macroscopic pores 110 to exhibit a larger surface area compared to other cross-sectional shapes may facilitate pulling the bodily fluids into the first porous layer 102 when the first porous layer 102 is relatively hydrophilic. However, cross-sectional shapes that cause the first macroscopic pores 110 to exhibit a smaller surface area compared to other cross-sectional shapes may facilitate the bodily fluids flowing through the first porous layer 102 when the first porous layer 102 is less hydrophilic.

As previously discussed, the porous material 100 includes the second porous layer 104. In an example, the second porous layer 104 is hydrophobic. The hydrophobicity of the second porous layer 104 prevents or at least inhibits bodily fluids that are present in the supporting layer 106 from flowing into the second porous layer 104. As such, the hydrophobicity of the second porous layer 104 generally maintains the bodily fluids in the supporting layer 106 (i.e., the layer that is configured to have the bodily fluids flow therein and therethrough). Further, the hydrophobicity of the second porous layer 104 generally repeals the bodily fluids thereby promoting the bodily fluids to flow through the supporting layer 106 and out of the porous material 100. Promoting the bodily fluids to flow through the supporting layer 106 causes the porous material 100 to dry quicker and pulls more of the bodily fluids from the first porous layer 102 into the supporting layer 106 due to a moisture gradient. The second porous layer 104 may be hydrophobic when the second porous layer 104 exhibits a contact angle with water that is about 90° or more, such as about 100° or more, about 110° or more, about 120° or more, about 130° or more, about 140° or more, about 150° of less, about 160° or more, about 170° or more, or in ranges of about 90° to about 110°, about 100° to about 120°, about 110° to about 130°, about 120° to about 140°, about 130° to about 150°, about 140° to about 160°, about 150° to about 170°, or about 160° to about 180°. Generally, increasing the hydrophobicity (i.e., increasing the contact angle with water) of the second porous layer 104 improves fluid flow through the porous material 100. In an embodiment, the first porous layer 102 is formed from a material (e.g., a hydrophilic material) that is treated to increase a hydrophobicity thereof or is coated with a hydrophobic material.

The second porous layer 104 may exhibit a thickness t₂ that is about 500 μm or less, such as about 400 μm or less, about 300 μm or less, about 250 μm or less, about 200 μm or less, about 150 μm or less, about 100 μm or less, or in ranges of about 50 μm to about 150 μm, about 100 μm to about 200 μm, about 150 μm to about 250 μm, about 200 μm to about 300 μm, about 250 μm to about 400 μm, or about 300 μm to about 500 μm. As previously discussed, the second porous layer 104 may be hydrophobic and at least inhibit bodily fluids flow therein which, in turn, decreases the volume of bodily fluids that may be temporarily stored in the porous material 100. Decreasing the volume of bodily fluids that may be temporarily stored in to the porous material 100 may increase the likelihood that the bodily fluids leak therefrom. As such, causing the second porous layer 104 to exhibit any of the relatively small thicknesses t₂ discussed above may cause the second porous layer 104 to have a minimal effect on the volume of bodily fluids that may be temporarily stored in the porous material 100. Also, the relatively small thickness t₂ may increase the overall thickness of the supporting layer 106 and the volume of bodily fluids that may flow therein over any given period of time.

The second porous layer 104 may define a plurality of second macroscopic pores 112 extending at least partially therethrough. The second macroscopic pores 112 include pores exhibiting a maximum dimension measured perpendicular to the longitudinal axis 108 that is about 1 mm or greater. The second macroscopic pores 112 may exhibit a second average macroscopic pore size D₂, measured perpendicularly to the longitudinal axis 108, that is about 1 mm or more, about 2 mm or more, about 3 mm or more, about 4 mm or more, about 5 mm or more, about 6 mm or more, about 7 mm or more, about 8 mm or more, about 9 mm or more, about 10 mm or more, or in ranges of about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, or about 8 mm to about 10 mm. The second average macroscopic pore size D₂ may be determined using the maximum dimension of the second macroscopic pores 112 or the mean dimension of the second macroscopic pores 112. It has been found that the presence of the second macroscopic pores 112 may improve flow of the bodily fluids through the supporting layer 106. However, generally, increasing the second average macroscopic pore size D₂ allows more bodily fluids to enter the second porous layer 104 since some of the bodily fluids in the second macroscopic pores 112 may not contact the hydrophobic layer of the second porous layer. Such bodily fluids that do not contact the second porous layer 104 may not be promoted to flow through the supporting layer 106, especially when the porous material 100 is relatively dry which inhibits drying the porous material 100. As such, increasing the second average macroscopic pore size D₂ may cause the porous material 100 to be slightly wetter.

The second macroscopic pores 112 may exhibit any suitable cross-sectional shape taken along a plane that is parallel to the longitudinal axis 108 and to an outer surface 111 of the second porous layer 104. For example, the second macroscopic pores 112 may exhibit a generally circular cross-sectional shape, generally rectangular (e.g., square) cross-sectional shape, a generally pentagonal cross-sectional shape, a generally hexagonal cross-sectional shape, a generally octagonal cross-sectional shape, a generally oval or ellipsoidal cross-sectional shape, a generally oblong cross-sectional shape, or any other suitable shape. The cross-sectional shape of the second macroscopic pores 112 may affect how effectively the second porous layer 104 repeals the bodily fluids when the second porous layer 104 is hydrophobic.

As previously discussed, the supporting layer 106 is positioned between the first and second porous layers 102, 104 and is configured to form a pathway for bodily fluids to flow. In an embodiment, the supporting layer 106 is formed from a plurality of fibers, such as a plurality of microfilaments. In an example, the plurality of fibers may be aligned in a first direction, wherein the first direction generally extends from the first porous layer 102 to the second porous layer 104 (e.g., aligned generally perpendicularly to the longitudinal axis 108). Aligning the fibers in the first direction allows the supporting layer 106 to more securely attach the first and second porous layers 102, 104 together. Further, the bodily fluids may be slightly more likely to flow in a direction that is parallel to the fibers. As such, aligning the fibers in the second direction may pull the bodily fluids through the supporting layer 106 quicker than if the fibers where otherwise oriented which causes the bodily fluids to flow in a greater percentage of the supporting layer 106 that if the bodily fluids where aligned in another direction. Causing the bodily fluids to flow in a greater percentage of the supporting layer 106 may cause a greater volume of the bodily fluids to flow through the porous material 100 at any given time and decrease the likelihood that the bodily fluids leak from the porous material 100.

In an embodiment, the supporting layer 106 may exhibit a percent void space that is greater than the percent void space of the first and second porous layers 102, 104. The percent void space refers to the volume of the layer that is unoccupied by a solid material divided by the total volume of the layer. The supporting layer 106 may exhibit a percent void space that is greater than the percent void space of the first and second porous layers 102, 104 by about 10% or more, about 20% or more, about 30% or more, about 40% or more, about 50% or more, about 75% or more, about 100% or more, about 150% or more, about 200% or more, about 250% or more, about 300% or more, about 400% or more, about 500% or more, or in ranges of about 10% to about 30%, about 20% to about 40%, about 30% to about 50%, about 40% to about 75%, about 50% to about 150%, about 100% to about 200%, about 150% to about 250%, about 200% to about 300%, about 250% to about 400%, or about 300% to about 500%. The increased percent void space of the supporting layer 106 relative to the first and second porous layers 102, 104 promotes flow of the bodily fluids therein. Promoting the flow of the bodily fluids through the supporting layer 106 may cause the supporting layer 106 to pull bodily fluids from the first porous layer 102 due to a moisture gradient and/or hydrogen bonding between water molecules, both of which may allow the first porous layer 102 to efficiently receive bodily fluids and to dry relatively quicker.

It has been surprisingly found that the supporting layer 106 may be formed from either a hydrophilic and/or hydrophobic material. In an example, the supporting layer 106 may be formed from a hydrophilic material exhibiting any of the hydrophilicities disclosed herein. As previously discussed, porous materials of conventional fluid collection assemblies are not formed of hydrophilic materials since such materials generally retain bodily fluids. However, the hydrophobicity of the second porous layer 104 limits the bodily fluids that may be retained by the supporting layer 106. Also, the increased percent void space limits the bodily fluids that may be held within supporting layer 106 and distributes the bodily fluids over a large surface area which promotes evaporation of the bodily fluids. When the supporting layer 106 is hydrophilic, the supporting layer 106 generally exhibits a hydrophilicity that is less than (e.g., a contact angle with water that is greater than) the first porous layer 102. In an example, the supporting layer 106 may be formed from a hydrophobic material exhibiting any of the hydrophobicities disclosed herein. In such an example, the supporting layer 106 may exhibit a hydrophobicity that is less than (e.g., a contact angle with water than is less than) the second porous layer 104 to promote the bodily fluids flowing from the first porous layer 102 to the supporting layer 106 and to allow the second porous layer 104 to repeal the bodily fluids.

The first porous layer 102, the second porous layer 104, and the supporting layer 106 may be formed from any suitable material. In an example, at least one of the first porous layer 102, the second porous layer 104, or the supporting layer 106 may be formed from one or more a polyester, polypropylene, nylon, cellulose, cotton, bamboo, or combinations thereof. In an example, at least one of the first porous layer 102, the second porous layer 104, or the supporting layer 106 may include a base material that is coated with a material. In such an example, the coating material may exhibit a hydrophilicity or hydrophobicity that is different than the base material. In an example, at least one of the first porous layer 102, the second porous layer 104, or the supporting layer 106 may be formed from at least one material that is treated to change a hydrophilicity or hydrophobicity thereof.

The porous material 100 may exhibit a thickness T measured from the first porous layer 102 to the second porous layer 104. The thickness T may be about 5 mm or greater, such as about 7.5 mm or greater, about 1 cm or greater, about 1.25 cm or greater, about 1.5 cm or greater, about 1.75 cm or greater, about 2 cm or greater, about 2.25 cm or greater, about 2.5 cm or greater, about 2.75 cm or greater, about 3 cm or greater, about 3.5 cm or greater, about 4 cm or greater, or in ranges of about 5 mm to 1 cm, about 7.5 mm to about 1.25 cm, about 1 cm to about 1.5 cm, about 1.25 cm to about 1.75 cm, about 1.5 cm to about 2 cm, about 1.75 cm to about 2.25 cm, about 2 cm to about 2.5 cm, about 2.25 cm to about 2.75 cm, about 2.5 cm to about 3 cm, about 2.75 cm to about 3.5 cm, or about 3 cm to about 4 cm. The thickness T of the porous material 100 may depend on the size of the chamber in which the porous material 100 is disposed, whether the porous material 100 is disposed in the chamber in a generally planar configuration or rolled into a generally cylindrical configuration, and the thickness of the first porous layer 102, the second porous layer 104, and the supporting layer 106.

The porous material 100 may be selected to exhibit a density of about 5 kg/m³ to about 10 kg/m³, about 7.5 kg/m³ to about 12.5 kg/m³, about 10 kg/m³ to about 15 kg/m³, about 12.5 kg/m³ to about 17.5 kg/m³, about 15 kg/m³ to about 20 kg/m³, about 17.5 kg/m³ to about 22.5 kg/m³, about 20 kg/m³ to about 25 kg/m³, about 22.5 kg/m³ to about 27.5 kg/m³, about 25 kg/m³ to about 30 kg/m³, about 27.5 kg/m³ to about 32.5 kg/m³, about 30 kg/m³ to about 35 kg/m³, about 32.5 kg/m³ to about 37.5 kg/m³, about 35 kg/m³ to about 37.5 kg/m³, about 35 kg/m³ to about 40 kg/m³, about 37.5 kg/m³ to about 42.5 kg/m³, about 40 kg/m³ to about 45 kg/m³, about 42.5 kg/m³ to about 47.5 kg/m³, or about 45 kg/m³ to about 50 kg/m³. Generally, increasing the density of the porous material 100 increases the strength of the porous material 100. However, increasing the density of the porous material 100 may decrease the porosity of the porous material 100 which decreases the volume of bodily fluids that may be temporarily stored in the porous material 100 and decrease the flow rate of the bodily fluids through the porous material 100. As such, the density of the porous material 100 may be selected based on balancing the desired strength, porosity, and flow rate of the bodily fluids through the porous material 100.

The porous material 100 may be selected to exhibit a basis weight of about 50 g/m² to about 100 g/m², about 75 g/m² to about 125 g/m², about 100 g/m² to about 150 g/m², about 125 g/m² to about 175 g/m², about 150 g/m² to about 200 g/m², about 175 g/m² to about 225 g/m², about 200 g/m² to about 250 g/m², about 225 g/m² to about 275 g/m², about 250 g/m² to about 300 g/m², about 275 g/m² to about 325 g/m², about 300 g/m² to about 375 g/m², about 350 g/m² to about 450 g/m², about 400 g/m² to about 500 g/m², about 450 g/m² to about 550 g/m², about 500 g/m² to about 600 g/m², about 550 g/m² to about 650 g/m², about 600 g/m² to about 700 g/m², about 650 g/m² to about 750 g/m², about 600 g/m² to about 700 g/m², about 650 g/m² to about 750 g/m², about 700 g/m² to about 800 g/m², about 750 g/m² to about 850 g/m², about 800 g/m² to about 900 g/m², about 850 g/m² to about 950 g/m², or about 900 g/m² to about 1000 g/m². The basis weight of the porous material 100 is a function of the density and thickness of the porous material 100. As such, the basis weight of the porous material 100 may be selected for any of the same reasons as the density and thickness of the porous material 100.

In an embodiment, as illustrated in FIG. 1, the first and second macroscopic pores 110, 112 of the first and second porous layers 102, 104, respectively, may be substantially the same. For example, the first and second average macroscopic pore sizes may be substantially similar and the cross-sectional shapes of the first and second macroscopic pores 110, 112 may be substantially the same. The first and second macroscopic pores 110, 112 may be substantially the same for a variety of reasons. In an example, the first and second macroscopic pores 110, 112 may be substantially the same when the first and second porous layers 102, 104 are formed from the same material except, for instance, at least one of the first and second porous layers 102, 104 may be coated or treated to exhibit different contact angles with water. In an example, selecting the first and second macroscopic pores 110, 112 to be substantially the same allows the first and second porous layers 102, 104 to be formed using the same processes except, for instance, different materials may be used in the process.

However, the first and second macroscopic pores of the porous material disclosed herein may be different. For example, FIG. 2 is a cross-sectional schematic of a porous material 200, according to an embodiment, that may be used in any of the fluid collection assemblies disclosed herein. Except as otherwise disclosed herein, the porous material 200 may be the same or substantially similar to any of the porous materials disclosed herein. For example, the porous material 200 may include a first porous layer 202, a second porous layer 204, and a supporting layer 206 between the first and second porous layers 202, 204.

The first porous layer 202 includes a plurality of first macroscopic pores 210 and the second porous layer 204 includes a plurality of second macroscopic pores 212. The first and second macroscopic pores 210, 212 are different from each other. In an embodiment, the first average macroscopic pore size D₁ of the first macroscopic pores 210 may be different than the second average macroscopic pore size D₂ of the second macroscopic pores 212. The first and second average macroscopic pore sizes D₁, D₂ may be selected to be different based on the desired properties for the reasons previously discussed. For example, as illustrated, the first average macroscopic pore size D₁ may be selected to be greater than the second average macroscopic pore size D₂. The larger first average macroscopic pore size D₁ may more efficiently receive the bodily fluids than if the first average macroscopic pore size D₁ was the same as the second average macroscopic pore size D₂. The smaller second average macroscopic pore size D₂ may allow the porous material 200 to be drier than if the second average macroscopic pore size D₂ was the same as the first average macroscopic pore size D₁. In an embodiment, the first macroscopic pores 210 may exhibit a cross-sectional shape that is different than the second macroscopic pores 212. The first and second macroscopic pores 210, 212 may be selected to exhibit different cross-sectional shapes for the reasons previously discussed.

Forming the first porous layer 202 and the second porous layer 204 to exhibit different average macroscopic pore sizes and/or different cross-sectional shapes may facilitate forming the fluid collection assemblies disclosed below. For instance, in some examples, the first and second porous layers of the porous materials disclosed herein may appear visually similar (e.g., similar color, similar texture, etc.). When the average macroscopic pore sizes and cross-sectional shapes of the pores of such visual similar first and second porous layers are the same, it may be difficult to correctly position the porous material in the fluid collection assemblies such that the first porous layer receives the bodily fluids before the second porous layer. Incorrectly positioning the porous material in the fluid collection assemblies may result in significant leakage of the bodily fluids. However, the different average macroscopic pore sizes and/or different cross-sectional shapes of the first and second porous layers 202, 204 make such porous layers easy to visual distinguish thereby facilitating formation of the fluid collection assemblies disclosed below.

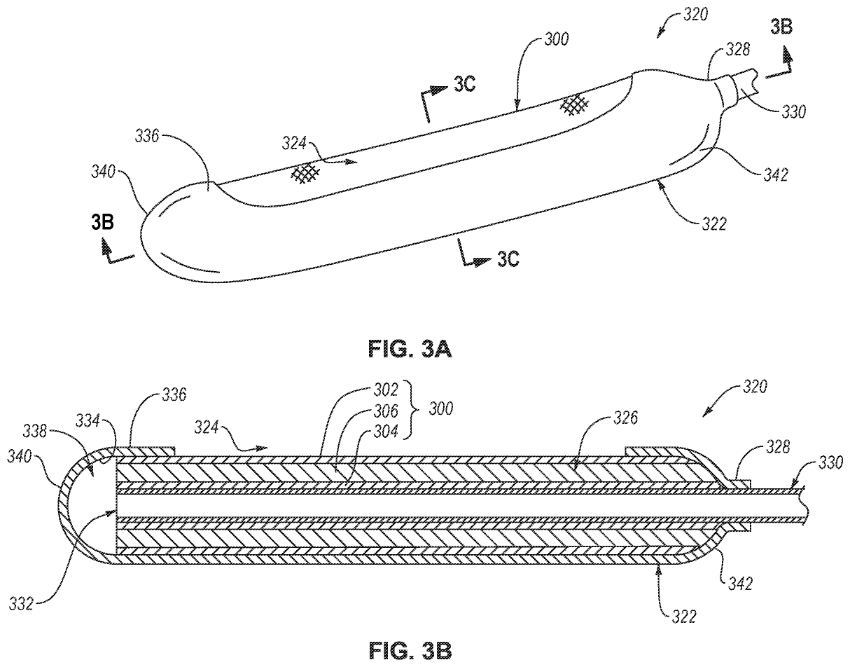

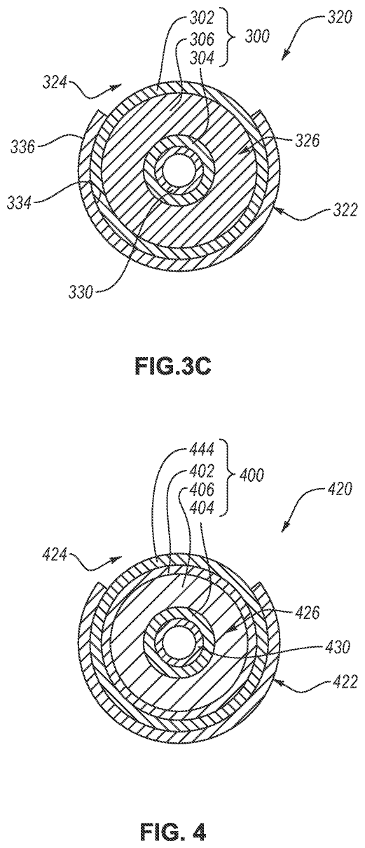

FIG. 3A is an isometric view of a fluid collection assembly 320 including a porous material 300, according to an embodiment. FIGS. 3B and 3C a cross-sectional schematics of the fluid collection assembly 320 taken along planes 3B-3B and 3C-3C, respectively, shown in FIG. 3A. The fluid collection assembly 320 is an example of a female fluid collection assembly for receiving and collecting bodily fluids from a female. The fluid collection assembly 320 includes a fluid impermeable layer 322 (e.g., fluid impermeable barrier) defining at least an opening 324, a chamber 326, and a fluid outlet 328. The fluid collection assembly 320 also includes the porous material 300 disposed in a chamber 326. The porous material 300 may be the same or substantially similar to any of the porous material disclosed herein. The fluid collection assembly 320 may further includes a conduit 330 is disposed through the fluid outlet 328 such that an inlet 332 of the conduit 330 is disposed in the chamber 326.

The fluid impermeable layer 322 at least partially defines a chamber 326 (e.g., interior region) and an opening 324. For example, the interior surface(s) 334 of the fluid impermeable layer 322 at least partially defines the chamber 326 within the fluid collection assembly 320. The fluid impermeable layer 322 temporarily stores the bodily fluids in the chamber 326. The fluid impermeable layer 322 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 322 substantially prevents the bodily fluids from passing through the fluid impermeable layer 322. In an example, the fluid impermeable layer 322 may be air permeable and fluid impermeable. In such an example, the fluid impermeable layer 322 may be formed of a hydrophobic material that defines a plurality of pores. At least one or more portions of at least an outer surface 336 of the fluid impermeable layer 322 may be formed from a soft and/or smooth material, thereby reducing chaffing.

In some examples, the fluid impermeable layer 322 may be tubular (ignoring the opening 324), such as substantially cylindrical (as shown), oblong, prismatic, or flattened tubes. During use, the outer surface 336 of the fluid impermeable layer 322 may contact the individual. The fluid impermeable layer 322 may be sized and shaped to fit between the labia and/or the gluteal cleft between the legs of a female user.

The opening 324 provides an ingress route for bodily fluids to enter the chamber 326. The opening 324 may be defined by the fluid impermeable layer 322 such as by an inner edge of the fluid impermeable layer 322. For example, the opening 324 is formed in and extends through the fluid impermeable layer 322, from the outer surface 336 322 to the inner surface 334, thereby enabling bodily fluids to enter the chamber 326 from outside of the fluid collection assembly 320.

The opening 324 may be an elongated hole in the fluid impermeable layer 322. For example, the opening 324 may be defined as a cut-out in the fluid impermeable layer 322. The opening 324 may be located and shaped to be positioned adjacent to a female urethral opening. The opening 324 may have an elongated shape because the space between the legs of a female is relatively small when the legs of the female are closed, thereby only permitting the flow of the bodily fluids along a path that corresponds to the elongated shape of the opening 324 (e.g., longitudinally extending opening 324).

The fluid collection assembly 320 may be positioned proximate to the female urethral opening and the bodily fluids may enter the chamber 326 of the fluid collection assembly 320 via the opening 324. The fluid collection assembly 320 is configured to receive the bodily fluids into the chamber 326 via the opening 324. When in use, the opening 324 may have an elongated shape that extends from a first location below the urethral opening (e.g., at or near the anus or the vaginal opening) to a second location above the urethral opening (e.g., at or near the top of the vaginal opening or the pubic hair).

In some examples, the fluid impermeable layer 322 may define a fluid outlet 328 sized to receive the conduit 330. The at least one conduit 330 may be disposed in the chamber 326 via the fluid outlet 328. The fluid outlet 328 may be sized and shaped to form an at least substantially fluid tight seal against the conduit 330 or the at least one tube thereby substantially preventing the bodily fluids from escaping the chamber 326.

As previously discussed, the porous material 300 is disposed in the chamber 326. The porous material 300 may be the same or substantially similar to any of the porous materials disclosed herein. For example, the porous material 300 may include a first porous layer 302, a second porous layer 304, and a supporting layer 306. The porous material 300 may be disposed in the chamber 326 such that the first porous layer 302 is positioned closer to the urethral opening of the individual than the second porous layer 304. For example, the first porous layer 302 may extend across the opening 324 and be exposed to an exterior of the fluid collection assembly 320. As such, the first porous layer 302 may contact the vaginal region of an individual when the fluid collection assembly 320 is positioned adjacent to a vaginal region. The porous material 300 may also be positioned such that the second porous layer 304 defines a bore that is configured to receive the conduit 330.

The porous material 300 may exhibit a generally cylindrical shape. In an embodiment, the porous material 300 may be provided exhibiting the generally cylindrical shape. In an embodiment, the porous material 300 may be provided in a sheet. In such an embodiment, the porous material 300 may be rolled into a generally cylindrical shape with opposing edges thereof contacting each other.

The porous material 300 may at least substantially completely fill the portions of the chamber 326 that are not occupied by the conduit 330. In some examples, the porous material 300 may not substantially completely fill the portions of the chamber 326 that are not occupied by the conduit 330. In such an example, the fluid collection assembly 320 includes the reservoir 338 disposed in the chamber 326.

The reservoir 338 is a substantially unoccupied portion of the chamber 326. The reservoir 338 may be defined between the fluid impermeable layer 322 and the porous material 300. The bodily fluids that are in the chamber 326 may flow through the fluid from the first porous layer 302 to the supporting layer 306 and through the supporting layer 306 to the reservoir 338. The reservoir 338 may retain of the bodily fluids therein.

The bodily fluids that are in the chamber 326 may flow through the supporting layer 306 to the reservoir 338. The fluid impermeable layer 322 may retain the bodily fluids in the reservoir 338. While depicted in the distal end region 340, the reservoir 338 may be located in any portion of the chamber 326 such as the proximal end region 342. The reservoir 338 may be located in a portion of the chamber 326 that is designed to be located in a gravimetrically low point of the fluid collection assembly when the fluid collection assembly is worn.

In some examples (not shown), the fluid collection assembly 320 may include multiple reservoirs, such as a first reservoir that is located at the portion of the chamber 326 closest to the inlet of the conduit 330 (e.g., distal end region 340) and a second reservoir that is located at the portion of the of the chamber 326 that is at or near proximal end region 342). In another example, the porous material 300 is spaced from at least a portion of the conduit 330, and the reservoir 338 may be the space between the porous material 300 and the conduit 330.

The conduit 330 may be at least partially disposed in the chamber 326. The conduit 330 may be used to remove the bodily fluids from the chamber 326. The conduit 330 includes at least one wall defining an inlet 332, an outlet (not shown) downstream from the inlet 332, and a passageway. The outlet of the conduit 330 may be operably coupled to a vacuum source, such as a vacuum pump for withdrawing fluid from the chamber 326 through the conduit 330. For example, the conduit 330 may extend into the fluid impermeable layer 322 from the proximal end region 342 and may extend to the distal end region 340 to a point proximate to the reservoir 338 therein such that the inlet 332 is in fluid communication with the reservoir 338. The conduit 330 fluidly couples the chamber 326 with the fluid storage container (not shown) or the vacuum source (not shown).

The conduit 330 may extend through a bore in the porous material 300 (e.g., a bore defined by the second porous layer 304). In an embodiment, the conduit 330 extends from the fluid outlet 328, through the bore, to a location that is proximate to the reservoir 338. In such an embodiment, the inlet 332 may not extend into the reservoir 338 and, instead, the inlet 332 may be disposed within the porous material 300 or at a terminal end thereof. In an embodiment, the conduit 330 is at least partially disposed in the reservoir 338 and the inlet 332 may be extended into or be positioned in the reservoir 338. The bodily fluids collected in the fluid collection assembly 320 may be removed from the chamber 326 via the conduit 330.

Locating the inlet 332 at or near a location expected to be the gravimetrically low point of the chamber 326 when worn by an individual enables the conduit 330 to receive more of the bodily fluids than if inlet 332 was located elsewhere and reduce the likelihood of pooling (e.g., pooling of the bodily fluids may cause microbe growth and foul odors). For instance, the bodily fluids in the supporting layer 306 may flow in any direction due to capillary forces. However, the bodily fluids may exhibit a preference to flow in the direction of gravity, especially when at least a portion of the supporting layer 306 is saturated with the bodily fluids. Accordingly, one or more of the inlet 332 or the reservoir 338 may be located in the fluid collection assembly 320 in a position expected to be the gravimetrically low point in the fluid collection assembly 320 when worn by an individual, such as the distal end region 340.

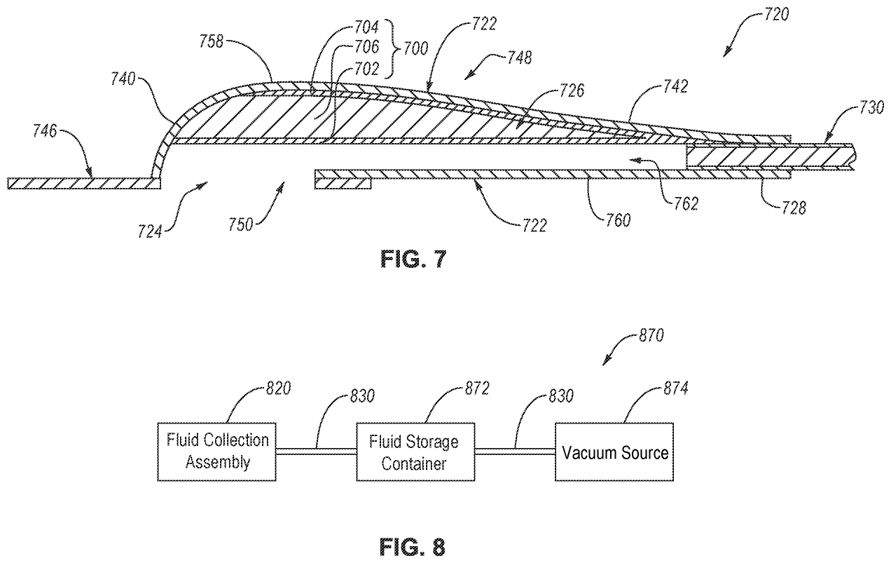

The inlet 332 and the outlet of the conduit 330 are configured to fluidly couple (e.g., directly or indirectly) the vacuum source (not shown) to the chamber 326 (e.g., the reservoir 338). As the vacuum source (FIG. 7) applies a vacuum/suction in the conduit 330, the bodily fluids in the chamber 326 (e.g., at the distal end region 340 such as in the reservoir 338) may be drawn into the inlet 332 and out of the fluid collection assembly 320 via the conduit 330. In some examples, the conduit 330 may be frosted or opaque (e.g., black) to obscure visibility of the bodily fluids therein.

As previously discussed, the conduit 330 may be configured to be at least insertable into the chamber 326. In an example, the conduit 330 may be positioned in the chamber 326 such that a terminal end of the conduit 330 is spaced from the fluid impermeable layer 322 or other components of the fluid collection assembly 320 that may at least partially obstruct or block the inlet 332. Further, the inlet 332 of the conduit 330 may be offset relative to a terminal end of the porous material 300 such that the inlet 332 is closer to the proximal end region 342 of the fluid collection assembly 320 than the terminal end of the porous material 300. Offsetting the inlet 332 in such a manner relative to the terminal end of the porous material 300 allows the inlet 332 to receive bodily fluids directly from the porous material 300 and, due to hydrogen bonding, pulls more bodily fluids from the porous material 300 into the conduit 330.

The porous materials disclosed herein may include one or more additional layers. For example, FIG. 4 is a cross-sectional schematic of a fluid collection assembly 420, according to an embodiment. Except as otherwise disclosed herein, the fluid collection assembly 420 is the same or substantially similar to any of the fluid collection assemblies disclosed herein. For example, the fluid collection assembly 420 includes a fluid impermeable layer 422 at least defining an opening 424 and a chamber 426. The fluid collection assembly 420 also includes a porous material 400 disposed in the chamber 426.

The porous material 400 includes a first porous layer 402, a second porous layer 404, and a supporting layer 406 disposed between the first and second porous layers 402, 404. The porous material 400 also includes a fluid permeable membrane 444. The fluid permeable membrane 444 is disposed on the first porous layer 402 and extends across the opening 424. The fluid permeable membrane 444 may improve the comfortability of the fluid collection assembly 420. For example, the first porous layer 402 may include a plurality of macroscopic pores (not shown). As previously discussed, the macroscopic pores of the first porous layer 402 may be uncomfortable against the vaginal region of the individual, especially when the macroscopic pores are large. The fluid permeable membrane 444 may be more comfortable against the vaginal region of the individual than the first porous layer 402. Thus, including the fluid permeable membrane 444 in the porous material 400 may make using the fluid collection assembly 420 more comfortable.

The fluid permeable membrane 444 may be composed to wick the bodily fluids away from the opening 424, thereby preventing the bodily fluids from escaping the chamber 426. In an embodiment, the fluid permeable membrane 444 may be configured to wick any bodily fluids away from the opening 424, thereby preventing the bodily fluids from escaping the chamber 426. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the fluid permeable membrane 444. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable membrane 444 (e.g., absorbency), such as less than about 30 wt % of the dry weight of the fluid permeable membrane 444, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable membrane 444. The fluid permeable membrane 444 may also wick the bodily fluids generally towards an interior of the chamber 426. In an embodiment, the fluid permeable membrane 444 may include at least one absorbent or adsorbent material. It is noted that including the fluid permeable membrane 444 in the porous material 400 may decrease how efficiently the porous material 400 receives bodily fluids and may increase the time that the porous material 400 remains wet.

In an embodiment, the fluid permeable membrane 444 may include any material that may wick the bodily fluids. For example, the fluid permeable membrane 444 may include fabric, such as a gauze (e.g., a silk, linen, or cotton gauze), another soft fabric, another smooth fabric, a nonwoven material, bamboo fibers, polypropylene fibers, cellulose fibers, any of the other porous materials disclosed herein, or combinations of any of the foregoing. Forming the fluid permeable membrane 444 from gauze, soft fabric, and/or smooth fabric may reduce chaffing caused by the fluid collection assembly 420.

The porous material 400 may include additional layers instead of or in addition to the fluid permeable membrane 444. In an embodiment, the porous material 400 may include a fluid permeable support configured to support the fluid permeable membrane 444 since the fluid permeable membrane 444 may be formed from a relatively foldable, flimsy, or otherwise easily deformable material. As such, the fluid permeable support may contact and extend inwardly from the fluid permeable membrane 444 (e.g., between the fluid permeable membrane 444 and the first porous layer 402 or the conduit 430). The fluid permeable support may be more rigid that the fluid permeable membrane 444 and may include, for example, porous polymer (e.g., nylon, polyester, polyurethane, polyethylene, polypropylene, etc.) structure or an open cell foam, such as spun nylon fiber. In an embodiment, the porous material 400 may include a foam, such as a polyurethane foam, polypropylene foam, or polyethylene foam. In an embodiment, at least one of the first porous layer 402, the second porous layer 404, or the supporting layer 406 may be omitted with the porous material 400 includes the one or more additional layers. The additional layers disclosed herein may be formed from any of the porous materials disclosed herein or any other suitable porous material. Further examples of porous materials that may form the one or more additional layers are disclosed in PCT International Application No. PCT/US2022/011281 filed on Jan. 5, 2022, PCT International Application No. PCT/US2022/042719 filed on Sep. 7, 2022, PCT International Application No. PCT/US2022/042725 filed on Sep. 7, 2022, U.S. Provisional Patent Application No. 63/241,564 filed on Sep. 8, 2021, PCT International Application No. PCT/US2022/015418 filed on Feb. 7, 2022, and PCT International Application No. PCT/US2022/015420 filed on Feb. 7, 2022, the disclosures of each of which are incorporated herein, in its entirety, by this reference.

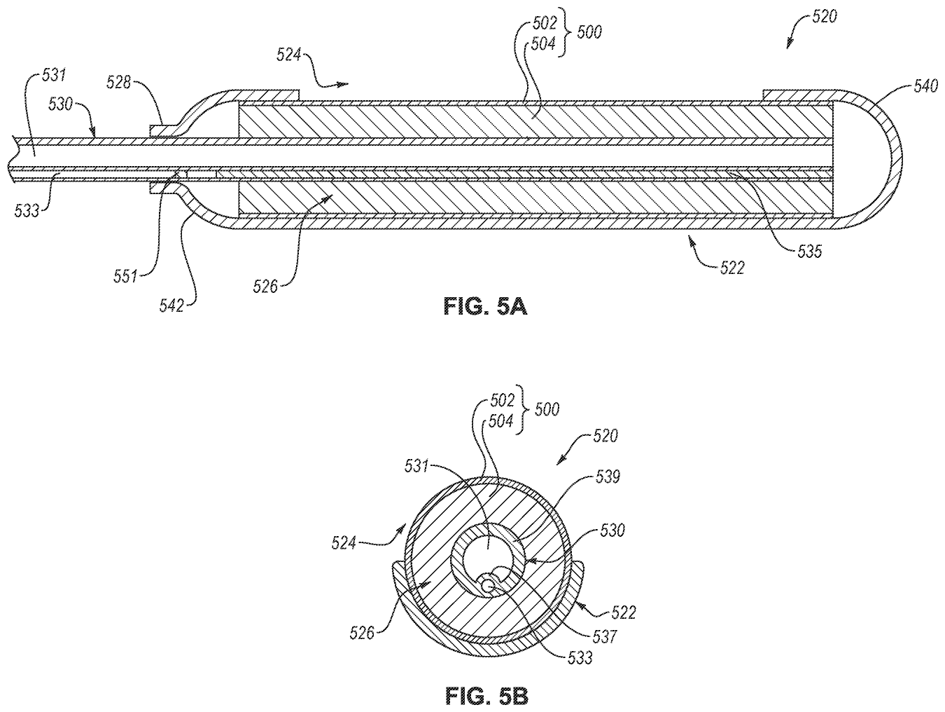

The fluid collection assemblies disclosed herein may include features (e.g., shape memory material) other than or in addition to a porous material including a first porous layer, a second porous layer, and a supporting layer. FIG. 5A is a cross-sectional view of a fluid collection assembly 520 including a shapeable conduit 530, according to an embodiment. FIG. 5B is a cross-sectional view of the fluid collection assembly 520 taken along plane 5B-5B, according to an embodiment. Except as otherwise disclosed herein, the fluid collection assembly 520 is the same as or substantially similar to any of the fluid collection assemblies disclosed herein. For example, the fluid collection assembly 520 includes a fluid impermeable layer 522 defining at least one opening 524, a chamber 526, and a fluid outlet 528. The fluid collection assembly 520 also includes at least one porous material 500 and a conduit 530.

The conduit 530 defines at least a first passageway 531 and a second passageway 533. The first passageway 531 is configured to remove bodily fluids from the chamber 526. For example, the first passageway 531 may be in fluid communication with a fluid storage container and a vacuum source (e.g., fluid storage container 872 and vacuum source 874 of FIG. 8) such that suction from the vacuum source may remove bodily fluids from the chamber 526 and deposit the bodily fluids in the fluid storage container via the first conduit 730. The second passageway 533 is configured to receive a shape memory material 535 and is not configured to receive bodily fluids from the chamber 526. As such, the second passageway 533 may not be in fluid communication with the fluid storage container or the vacuum source. The second passageway 533 may exhibit a cross-sectional area that is smaller (e.g., at least 5 times smaller or at least 10 times smaller) than the cross-sectional area of the first passageway 531 since the second passageway 533 is not configured to receive bodily fluids from the chamber 526.

The first and second passageways 531, 533 are distinct from each other. For example, the conduit 530 may include an interior wall 537 that separates the first and second passageways 531, 533 from each other. The interior wall 537 may be a fluid impermeable material and may be integrally formed (e.g., exhibit single piece construction) with the rest of the conduit 530. In an embodiment, the conduit 530 includes an exterior wall 539 exhibiting a generally cylindrical shape which allows the conduit 530 to be used with fluid collection assemblies that are configured to use a cylindrical conduit. In such an embodiment, the interior wall 537 may extend inwardly from the exterior wall 539 which may cause the first passageway 531 to exhibit a generally crescent shape.

As previously discussed, the conduit 530 includes a shape memory material 535. The shape memory material 535 may be sized, shaped, and positioned in the conduit 530 to cause at least a portion of the conduit 530 to retain a selected shape (e.g., geometric configuration). Since the conduit 530 is at least partially disposed in the chamber 526, the selected shape also causes the rest of the fluid collection assembly 520 to exhibit a similar shape. In an embodiment, the shape memory material 535 is configured to be bent, shaped, or otherwise deformed (hereafter collectively referred to as “shape.” “shaped.” or “shaping”). In an example, the shape memory material 535 is configured to be shaped along an entire length thereof. Allowing the shape memory material 535 to be shaped along the entire length thereof may allow the fluid collection assembly 520 to exhibit a shape that substantially corresponds to the anatomical features of the wearer. In an example, the shape memory material 535 is configured to be shaped at one or more selected locations thereof. In such an example, the selected locations of the shape memory material 535 may be preferentially shaped relative to the rest of the shape memory material 535. While configuring the shape memory material 535 to be shaped at the selected locations may inhibit the fluid collection assembly 520 from exhibiting a shape that substantially corresponds to the anatomical features of the wearer, it may facilitate shaping of the fluid collection assembly 520, especially for less experienced wearers. In an embodiment, the shape memory material 535 may not be configured to be shaped. Instead, the shape memory material 535 may exhibit a selected shape that corresponds or substantially corresponds to the anatomical feature of the wearer. In such an embodiment, the shape memory material 535 may be more rigid and/or resilient than the rest of the fluid collection assembly 520 thereby causing at least a portion of the fluid collection assembly 520 to correspond to the selected shape of the shape memory material 535.

The shape memory material 535 may include a shape memory polymer or a metal (e.g., shape memory metal). Generally, the shape memory material 535 is composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory material 535 herein may simply hold a selected shape when bent, set, or cured into a specific shape and/or when cooled in a specific shape, regardless of the stimuli applied thereto after. The shape memory material 535 may be returned to the original shape or changed to a new shape by application of stimuli. For example, a metal wire bent to a first shape may be utilized as the shape memory material 535, whereinafter the metal wire may be modified to a second shape via physical force applied thereto or via heating. However, in some embodiments, the shape memory material 535 may exhibit a selected shape, as discussed above and application of the stimuli may cause the shape memory material 535 to deform (e.g., elastically deform or bend) into an intermediate shape. In such embodiments, the shape memory material 535 may return to the initial shape upon removal of the stimuli such that the shape memory material 535 does not maintain the intermediate shape.

In an embodiment, the shape memory material 535 may include a shape memory metal, such as an elemental metal, an alloy, or shape memory alloy. Suitable shape memory metals may include standard steels, stainless steel, carbon alloy steel, head treated steel, aluminum, silver. copper, iron, nickel, zinc, tin, beryllium, or the like. Suitable shape memory alloys may include stainless steel; galvanized steel: aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn. or the like; Co—Cr—Ni—Mo alloys (e.g., Elgiloy®) or the like; or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be at least partially coated with a polymer (e.g., polyvinyl chloride), anodized, passivated, or otherwise treated to prevent corrosion. At least partially coating the shape memory metal with a polymer may also prevent metal ions from the shape memory material 535 from entering the chamber 526 and contacting the vaginal region which may cause irritation.

Shape memory polymers (“SMPs”) may include polyurethane-based SMPs such as a copolymer (e.g., copolyester, polyurethane, polyetherester, etc.) including blocks of one or more of poly(s-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polypropylene, polyethylene, acrylonitrile butadiene styrene (ABS), polysulphone, or the like; polynorbonene; other deformable polymers; or any other shape memory polymer.

The fluid collection assembly 520 may be shaped to contour to the anatomy of a wearer using the fluid collection assembly 520 to improve comfort over conventional devices and to remain in position during use. The fluid collection assembly 520 may be manipulated to contour to the anatomy in the groin region of a wearer. For example, the conduit 530 may be shaped upwardly such that the fluid collection assembly 520 maintains a generally arcuate shape. In such examples, the distal end region 540 may be positioned in the gluteal cleft of the wearer, the proximal end region 542 may be positioned against the upper vaginal or pubic area of the wearer, and the portion therebetween may be shaped to contour the anatomy of the wearer. The shape in the fluid collection assembly 520 may be more or less arcuate depending on the size and shape of the wearer. Accordingly, the fluid collection assembly 520 may be utilized with a variety of differently sized wearers.

The shape memory material 535 includes at least one wire (e.g., at least one rod). The wire includes a length measured along a longitudinal axis of the wire, a width measured perpendicularly to the length, and a thickness measured perpendicularly to the length and the width. The length of the wire is significantly greater than the width and the thickness. In an embodiment, the wire is sized and configured such that the length is generally aligned with the longitudinal axis of the fluid impermeable layer 522. In such an embodiment, the wire can change the shape of the fluid collection assembly 520 along the longitudinal axis thereof and/or change the shape of the fluid impermeable layer 522 globally. In an embodiment, the wire is sized and configured to such that the length is not aligned with the longitudinal axis of the fluid impermeable layer 522.

The length of the wire may be at least 10% of the longitudinal length of the conduit 530 that is disposed in the chamber 526, such as 10% to 100%, 30% to 100%, 10% to 40%, 30% to 60%, 60% to 90%, 40% to 80%, 50% to 100%, less than 100%, less than 70%, or greater than 100% of the length of the conduit 530 that is disposed in the chamber 526. It is noted that selecting the length of the wire to be substantially equal to or greater than the length of the conduit 530 that is disposed in the chamber 526 allows the shape of the fluid collection assembly 520 to be changed globally. In an example, the wire may exhibit a generally circular cross-sectional shape.

In an embodiment, the conduit 530 includes a plug 551 disposed in the second passageway 533. The plug 551 may prevent over insertion of the shape memory material 535 into the second passageway 533 since over insertion of the shape memory material 535 into the second passageway 533 may prevent shaping of one or more desired regions of the fluid collection assembly 520. In an example, the plug 551 may be disposed at or near the fluid outlet 528 thereby allowing the shape memory material 535 to be disposed in substantially all of the length of the conduit 530 which, in turn, may allow the shape memory material 535 to affect the global shape of the fluid collection assembly 520. The plug 551 may also form a substantially fluid tight seal in the second passageway 533 thereby preventing the flow of bodily fluids through the second passageway 533.

As previously discussed, the fluid collection assembly 520 includes a porous material 500 disposed in the chamber 526. In an embodiment, not shown, the porous material 500 includes the porous material illustrated in FIGS. 1-4, that is, a first porous layer, a second porous layer, and a supporting layer disposed therebetween. In an embodiment, the porous material 500 includes a fluid permeable outer layer 502 and a fluid permeable inner layer 504. In an example, the outer layer 502 may be thinner than the inner layer 504. For example, the outer layer 502 may exhibit a thickness of about 0.1 mm to about 0.5 mm and the inner layer 504 may exhibit a thickness of about 5 mm to about 10 mm.

The outer layer 502 and the inner layer 504 may include any of the porous materials disclosed herein. In an example, the outer layer 502 and/or the inner layer 504 may include the porous material illustrated in FIGS. 1-4, namely a first porous layer, a second porous layer, and a supporting layer therebetween. In an example, the outer layer 502 and/or the inner layer 504 includes one or two of the first porous layer, the second porous layer, or the supporting layer. In an example, the outer layer 502 is a fluid permeable membrane. In such an example, the outer layer 502 may include gauze, bamboo fibers, polypropylene fibers, cellulose fibers, any of the other fluid permeable membranes disclosed herein, or combinations of any of the foregoing. When the outer layer 502 is a fluid permeable membrane, the outer layer 502 may exhibit a density of about 25 g/m² to about 100 g/m² since it has been found that an outer layer 502 exhibiting such densities may efficiently receive bodily fluids from the wearer. However, it is noted that the fluid permeable membrane of the outer layer 502 may exhibits densities below 25 g/m² or greater than 100 g/m². In an example, the inner layer 504 may be a fluid permeable support. In such an example, the inner layer 504 may include a foam, any of the other fluid permeable supports disclosed herein, or a combination of any of the foregoing. When the inner layer 504 is a fluid permeable support, the inner layer 504 may exhibit a density of about 100 g/m² to about 350 g/m² since it has been found that an inner layer 504 exhibiting such densities may allow the bodily fluids to efficiently flow therethrough. However, it is noted that the fluid permeable support of the inner layer 504 may exhibits densities below 100 g/m² or greater than 350 g/m². In an embodiment, the porous material 500 may include a single material.

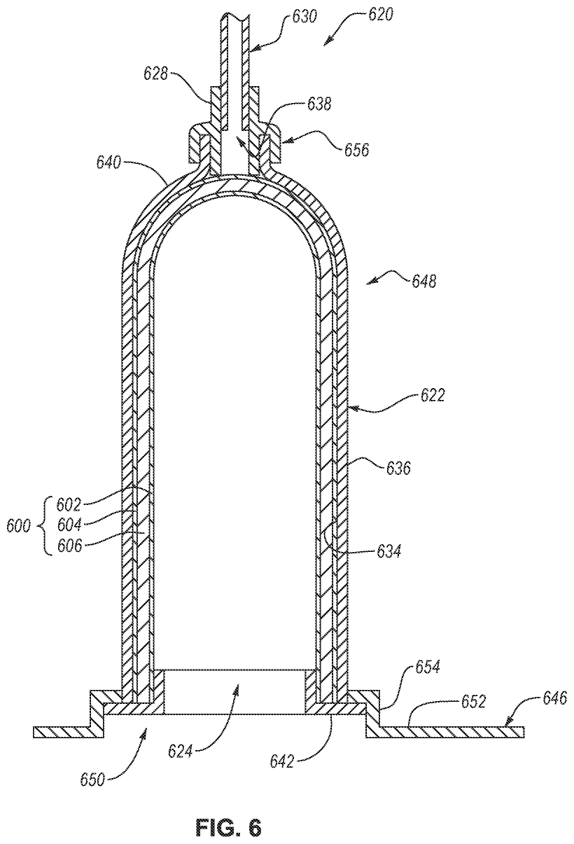

The fluid collection assemblies shown in FIGS. 3A-4 are examples of female fluid collection assemblies that are configured to collect bodily fluids from females (e.g., collect urine from a female urethra). However, the fluid collection assemblies, systems, and methods disclosed herein may include male fluid collection assemblies shaped, sized, and otherwise configured to collect bodily fluids from males (e.g., collect urine from a male urethra). FIG. 6 is a cross-sectional view of a male fluid collection assembly 620, according to an embodiment.

The fluid collection assembly 620 includes a base 646 (e.g., annular base) and a sheath 648. The base 646 is sized, shaped, and made of a material to be coupled to skin that surrounds the male urethral opening (e.g., penis) and have the male urethral opening positioned therethrough. For example, the base 646 may define an aperture 650. The base 646 is sized and shaped to be positioned around the male urethral opening (e.g., positioned around and/or over the penis) and the aperture 650 may be configured to have the male urethral opening positioned therethrough. The base 646 may also be sized, shaped, made of a material, or otherwise configured to be coupled (e.g., adhesively attached, such as with a hydrogel adhesive) to the skin around the male urethral opening (e.g., around the penis). In an example, the base 646 may exhibit the general shape or contours of the skin surface that the base 646 is selected to be coupled with. The base 646 may be flexible thereby allowing the base 646 to conform to any shape of the skin surface. The base 646 may include a laterally (e.g., radially) extending flange 652. The base 646 also defines a hollowed region that is configured to receive (e.g., seal against) the sheath 648. For example, the base 646 may include a longitudinally extending flange 654 that extends upwardly from the base 646. The longitudinally extending flange 654 may be tall enough to prevent the sheath 648 from being accidentally removed from the base 646 (e.g., at least 0.25 cm tall, 1 cm tall, at least 3 cm tall, or at least 5 cm tall). The base 646 is located at a proximal end region 642 (with respect to a wearer) of the fluid collection assembly 620.

The sheath 648 includes (e.g., may be formed from) a fluid impermeable layer 622 that is sized and shaped to fit into the hollowed region of the base 646. For example, the sheath 648 may be generally tubular or cup-shaped, as shown. The generally tubular or cup-shaped fluid impermeable layer 622 may at least partially define the outer surface 636 of the sheath 648. The fluid impermeable layer 622 may be similar or identical to and of the fluid impermeable layers disclosed herein, in one or more aspects. For example, the fluid impermeable layer 622 may be constructed of any of the materials disclosed herein for the fluid impermeable layer. The fluid impermeable layer 622 at least partially defines the chamber 626. For example, the inner surface 634 of the fluid impermeable layer 622 at least partially defines the perimeter of the chamber 626. The chamber 626 may at least temporarily retain bodily fluids therein. As shown, the fluid collection assembly 620 may include the porous material 600 therein. The porous material 600 may be similar or identical any of the porous materials disclosed herein, in one or more aspects. For example, the porous material 600 may include a first porous layer 602, a second porous layer 604, and a supporting layer 606 disposed between the first and second porous layer 602. Optionally, the porous material 600 may include a fluid permeable membrane (not shown) disposed on the first porous layer 602 such that the fluid permeable membrane contacts the penis disposed in the chamber 626 to improve comfort. The fluid impermeable layer 622 may also define an opening 624 extending through the fluid impermeable layer 622 that is configured to have a male urethral opening positioned therethrough.

The sheath 648 also may include at least a portion of the conduit 630 therein, such as the conduit 630 at least partially disposed in the chamber 626. For example, not shown, the conduit 630 may extend from the sheath 648 at the distal end region 640 to a proximal end region 642 at least proximate to the opening 624. The proximal end region 642 may be disposed near or on the skin around the male urethral opening (e.g., on the penis or pubic area therearound). Accordingly, when an individual lays on their back, bodily fluids (e.g., urine) may aggregate near the opening 624 against the skin of the subject. The bodily fluids may be removed from the chamber 626 via the conduit 630.

In some examples, the fluid impermeable layer 622 may be constructed of a material and/or have a thickness that allows the sheath 648 to collapse when placed under vacuum, such as to remove air around a penis in the fluid collection assembly 620 during use. In such examples, the conduit 630 may extend only to or into the distal end region 640 in the chamber 626 (e.g., not through to the area adjacent the opening 624). In such examples, urine may be collected and removed from the fluid collection assembly 620

In an example, portions of the chamber 626 may be substantially empty due to the varying sizes and rigidity of the male penis. However, in some examples, the outermost regions of the chamber 626 (e.g., periphery of the interior regions of the sheath 648) may include porous material 600. For example, the porous material 600 may be bonded to the inner surface 634 of the fluid impermeable layer 622. The porous material 600 may be positioned (e.g., at the distal end of the chamber 626) to blunt a stream of urine from the male urethral opening thereby limiting splashing and/or to direct the bodily fluids to a selected region of the chamber 626. Since the chamber 626 is substantially empty (e.g., substantially all of the chamber 626 forms a reservoir), the bodily fluids are likely to pool at a gravimetrically low point of the chamber 626. The gravimetrically low point of the chamber 626 may be at an intersection of the skin of an individual and the fluid collection assembly 620, a corner formed in the sheath 648, or another suitable location depending on the orientation of the wearer.

The porous material 600 may be disposed between the fluid impermeable layer 622 and a penis inserted into the chamber 626. The first porous layer 602 may be positioned between the fluid impermeable layer 622 and a penis inserted into the chamber 626, such as between the second porous layer 604 and penis and between the supporting layer 606 and the penis. The inner surface 634, optionally including the end of the chamber 626 substantially opposite the opening 624, may be covered with the second porous layer 604. The second porous layer 604 may be affixed (e.g., adhered) to the fluid impermeable layer 622.

The fluid collection assembly 620 includes a cap 656 at a distal end region 640. The cap 656 defines an interior channel through which the bodily fluids may be removed from the fluid collection assembly 620. The interior channel is in fluid communication with the chamber 626. The cap 656 may be disposed over at least a portion of the distal end region 640 of one or more of the fluid impermeable layer 622 or the porous material 600. The cap 656 may be made of a polymer, rubber, or any other fluid impermeable material. The cap 656 may be attached to one or more of the fluid impermeable layer 622, the porous material 600, or the conduit 630. The cap 656 may cover at least a portion of the distal end region 640 of the fluid collection assembly 620. The cap 656 may define a fluid outlet 628 that is sized and configured to receive and fluidly seal against the conduit 630. The conduit 630 may extend a distance within or through the cap 656, such as to the porous material 600, through the porous material 600, or to a point set-off from the porous material 600. In the latter example, the interior channel of the cap 656 may define a reservoir 638 therein.

The reservoir 638 is an unoccupied portion of device such as in the cap 656 and is void of other material. In some examples, the reservoir 638 is defined at least partially by the porous material 600 and the cap 656. During use, the bodily fluids that are in the chamber 626 may flow through the porous material 600 to the reservoir 638. The reservoir 638 may store at least some of the bodily fluids therein and/or position the bodily fluids for removal by the conduit 630. In some examples, at least a portion of the porous material 600 may extend continuously between at least a portion of the opening of the interior channel and chamber 626 to wick any bodily fluids from the opening directly to the reservoir 638.

In some examples (not shown), the fluid impermeable layer 622 may be disposed on or over the cap 656, such as enclosing the cap 656 within the chamber 626.

The proximal end region 642 may be disposed near or on the skin around the male urethral opening (e.g., around the penis) and the inlet of the conduit 630 may be positioned in the proximal end region 642. The outlet of the conduit 630 may be directly or indirectly coupled to a vacuum source. Accordingly, bodily fluids may be removed from the proximal end region 642 of the chamber 626 via the conduit 630.

The base 646, the sheath 648, the cap 656, and the conduit 630 may be attached together using any suitable method. For example, at least two of the base 646, the sheath 648, the cap 656, or the conduit 630 may be attached together using at least one of an interference fit, an adhesive, stitching, welding (e.g., ultrasonic welding), tape, any other suitable method, or combinations thereof.

In some examples (not shown), the fluid collection assembly 620 may have a one piece design, with one or more of the sheath 648, the base 646, and the cap 656 being a single, integrally formed piece.

Also as shown, the conduit 630 may be at least partially disposed with the chamber of a fluid collection assembly. The conduit 630 may extend from the distal end region 640 to the proximal end region 642. For example, the conduit 630 may extend through the cap 656 to a point adjacent to the base 646. The conduit 630 is sized and positioned to be coupled to a fluid storage container or the vacuum source (FIG. 8). An outlet of the conduit 630 may be operably coupled to the vacuum source, directly or indirectly. The inlet 632 of the conduit 630 may be positioned within the chamber 626 such as at a location expected to be at the gravimetrically low point of the fluid collection assembly 620 during use. By positioning the inlet 632 in a location expected to be at the gravimetrically low point of the fluid collection assembly when worn by the user, bodily fluids introduced into the chamber 626 may be removed via the conduit 630 to prevent pooling or stagnation of the bodily fluids within the chamber 626.

In some examples, the vacuum source may be remotely located from the fluid collection assembly 620. In such examples, the conduit 630 may be fluidly connected to the fluid storage container, which may be disposed between the vacuum source and the fluid collection assembly 620.

During operation, a male using the fluid collection assembly 620 may discharge bodily fluids (e.g., urine) into the chamber 626. The bodily fluids may pool or otherwise be collected in the chamber 626. At least some of the bodily fluids may be pulled through the interior of the conduit 630 via the inlet. The bodily fluids may be drawn out of the fluid collection assembly 620 via the vacuum/suction provided by the vacuum source. During operation, a vacuum relief valve (not shown) may substantially maintain the pressure in the chamber 626 at atmospheric pressure even though bodily fluids is introduced into and subsequently removed from the chamber 626.

FIG. 7 is a cross-sectional view of a fluid collection assembly 720, according to an embodiment. The fluid collection assembly 720 is an example of a male fluid collection assembly though, in some embodiments, the fluid collection assembly 720 may be used to receive bodily fluids from a female urethral opening. Except as otherwise disclosed herein, the fluid collection assembly 720 is the same or substantially similar to any of the fluid collection assemblies disclosed herein. The fluid collection assembly 720 includes a sheath 748 and a base 746. The base 746 is configured to be attached (e.g., permanently attached to or configured to be permanently attached) to the sheath 748. The base 746 is also configured to be attached to the region about the urethral opening (e.g., penis) of the individual.

The sheath 748 includes a fluid impermeable layer 722 that is at least partially formed from a first panel 758 and a second panel 760. The first panel 758 and the second panel 760 may be attached or integrally formed together (e.g., exhibits single piece construction). In an embodiment, as illustrated, the first panel 758 and the second panel 760 are distinct sheets. The fluid impermeable layer 722 also defines a chamber 726 between the first panel 758 and the second panel 760, an opening 724 at a proximal end region 742 of the sheath 748, and a fluid outlet 728 at a distal end region 740 of the sheath 748. The sheath 748 also includes at least one porous material 700 disposed in the chamber 726.

The inner surface(s) of the fluid impermeable layer 722 (e.g., inner surfaces of the first and second panels 758, 760 at least partially defines the chamber 726 within the fluid collection assembly 720. The fluid impermeable layer 722 temporarily stores the bodily fluids in the chamber 726. The fluid impermeable layer 722 may be formed from any of the fluid impermeable materials disclosed herein. As such, the fluid impermeable layer 722 substantially prevents the bodily fluids from passing through the fluid impermeable layer 722.

In an embodiment, at least one of the first panel 758 or the second panel 760 is formed from an at least partially transparent fluid impermeable material, such as polyethylene, polypropylene, polycarbonate, or polyvinyl chloride. Forming at least one of the first panel 758 or the second panel 760 from an at least partially transparent fluid impermeable material allows a person (e.g., medical practitioner) to examiner the penis. In some embodiments, both the first panel 758 and the second panel 760 are formed from at least partially transparent fluid impermeable material. Selecting at least one of the first panel 758 or the second panel 760 to be formed from an at least partially transparent impermeable material allows the penis to be examined without detaching the entire fluid collection assembly 720 from the region about the penis. For example, the chamber 726 may include a penis receiving area 762 that is configured to receive the penis of the individual when the penis extends into the chamber 726. The penis receiving area 762 may be defined by at least the porous material 700 and at least a portion of the at least partially transparent material of the first panel 758 and/or the second panel 760. In other words, the porous material 700 is positioned in the chamber 726 such that the porous material 700 is not positioned between the penis and at least a portion of the transparent portion of the first panel 758 and/or second panel 760 when the penis is inserted into the chamber 726 through the opening 724. The porous material 700 is generally not transparent and, thus, the portion of the at least partially transparent material of the first panel 758 and/or the second panel 760 that defines the penis receiving area 762 forms a window which allows the person to view into the penis receiving area 762 and examine the penis.

The opening 724 defined by the fluid impermeable layer 722 provides an ingress route for bodily fluids to enter the chamber 726 when the penis is a buried penis and allow the penis to enter the chamber 726 (e.g., the penis receiving area 762) when the penis is not buried. The opening 724 may be defined by the fluid impermeable layer 722 (e.g., an inner edge of the fluid impermeable layer 722). For example, the opening 724 is formed in and extends through the fluid impermeable layer 722 thereby enabling bodily fluids to enter the chamber 726 from outside of the fluid collection assembly 720.

The fluid impermeable layer 722 defines the fluid outlet 728 sized to receive the conduit 730. The conduit 730 may be at least partially disposed in the chamber 726 or otherwise in fluid communication with the chamber 726 through the fluid outlet 728. The fluid outlet 728 may be sized and shaped to form an at least substantially fluid tight seal against the conduit 730 thereby substantially preventing the bodily fluids from escaping the chamber 726. In an embodiment, the fluid outlet 728 may be formed from a portion of the first panel 758 and the second panel 760 that are not attached or integrally formed together. In such an embodiment, the fluid impermeable layer 722 may not include a cap exhibiting a rigidity that is greater than the portions of the fluid impermeable layer 722 thereabout which may facilitate manufacturing of the fluid collection assembly 720 by decreasing the number of parts that are used to form the fluid collection assembly 720. The lack of the cap may make it difficult to secure the conduit 730 to the fluid outlet 728 using an interference fit. As such, the conduit 730 may be attached to the fluid outlet 728 (e.g., to the first and second panels 758, 760) using an adhesive, a weld, or otherwise bonding the fluid outlet 728 to the fluid outlet 728. In an example, the conduit 730 may be attached to the fluid outlet 728 in the same manufacturing step that attaches the first and second panels 758, 760 together. In an example, the fluid impermeable layer 722 includes a cap and the conduit 728 is attached (e.g., via an interference fit) to the cap.

As previously discussed, the sheath 748 includes at least one porous material 700 disclosed in the chamber 726. The porous material 700 may direct the bodily fluids to one or more selected regions of the chamber 726, such as away from the penis and towards the fluid outlet 728. The porous material 700 may be formed from any of the porous materials disclosed herein. For example, the porous material 700 may include a first porous layer 702, a second porous layer 704, and a supporting layer 706 between the first and second porous layers 702, 704. In an embodiment, the first porous layer 702 may be positioned to at least a portion define the penis receiving area 762. The second porous layer 704 may be positioned adjacent to the first panel 758.

In an embodiment, the porous material 700 may be a sheet. Forming the porous material 700 as a sheet may facilitate the manufacturing of the fluid collection assembly 720. For example, forming the porous material 700 as a sheet allows the first panel 758, the second panel 760, and the porous material 700 to each be sheets. During the manufacturing of the fluid collection assembly 720, the first panel 758, the second panel 760, and the porous material 700 may be stacked and then attached to each other in the same manufacturing step. For instance, the porous material 700 may exhibit a shape that is the same size or, more preferably, slightly smaller than the size of the first panel 758 and the second panel 760. As such, attaching the first panel 758 and the second panel 760 together along the outer edges thereof may also attach the porous material 700 to the first panel 758 and the second panel 760. The porous material 700 may be slightly smaller than the first panel 758 and the second panel 760 such that the first panel 758 and/or the second panel 760 extend around the porous material 700 such that the porous material 700 does not form a passageway through the fluid impermeable layer 722 through which the bodily fluids may leak. Also, attaching the porous material 700 to the first panel 758 and/or the second panel 760 may prevent the porous material 700 from significantly moving in the chamber 726, such as preventing the porous material 700 from bunching together near the fluid outlet 728. In an example, the porous material 700 may be attached to the first panel 758 or the second panel 760 (e.g., via an adhesive) before or after attaching the first panel 758 to the second panel 760. In an example, the porous material 700 may merely be disposed in the chamber 726 without attaching the porous material 700 to at least one of the first panel 758 or the second panel 760. In an embodiment, the porous material 700 may exhibit shapes other than a sheet, such as a hollow generally cylindrical shape.

Generally, the sheath 748 is substantially flat when the penis is not in the penis receiving area 762 and the sheath 748 is resting on a flat surface. The sheath 748 is substantially flat because the fluid impermeable layer 722 is formed from the first panel 758 and the second panel 760 instead of a generally tubular fluid impermeable layer. Further, as previously discussed, the porous material 700 may be a sheet, which also causes the sheath 748 to be substantially flat. The sheath 748 may also be substantially flat because the fluid collection assembly 720 may not include relatively rigid rings or caps that exhibit a rigidity that is greater than the portions of the fluid impermeable layer 722 thereabout since such rings and caps may inhibit the sheath 748 being substantially flat. It is noted that the sheath 748 is described as being substantially flat because at least one of the porous material 700 may cause a slight bulge to form in the sheath 748 depending on the thickness of the porous material 700, the fluid outlet 728 and/or conduit 730 may cause a bulge thereabout, or the base 746 may pull on portions of the sheath 748 thereabout. It is also noted that the sheath 748 may also be compliant and, as such, the sheath 748 may not be substantially flat during use since, during use, the sheath 748 may rest on a non-flat surface (e.g., may rest on the testicles, the perineum, and/or between the thighs) and the sheath 748 may conform to the surface of these shapes.

The ability of the sheath 748 to be substantially flat when the penis is not in the penis receiving area 762 and the sheath 748 is resting on a flat surface allows the fluid collection assembly 720 to be used with a buried and a non-buried penis. For example, when the fluid collection assembly 720 is being used with a buried penis, the penis does not extend into the penis receiving area 762 which causes the sheath 748 to lie relatively flat across the aperture 750 of the base 746. When the sheath 748 lies relatively flat across the aperture 750 of the base 746, the porous material 700 extends across the opening 724 and the aperture 750 and is in close proximity to the buried penis. As such, the porous material 700 prevents or inhibits pooling of bodily fluids discharged from the buried penis against the skin of the individual since the porous material 700 will receive and remove at least a significant portion of the bodily fluids that would otherwise pool against the skin of the individual. Thus, the skin of the individual remains dry thereby improving comfort of using the fluid collection assembly 720 and preventing skin degradation. However, unlike other conventional fluid collection assemblies that are configured to be used with buried penises, the fluid collection assembly 720 may still be used with a non-buried penis since the non-buried penis can still be received into the penis receiving area 762, even when the penis is fully erect. Additionally, the ability of the sheath 748 to be substantially flat allows the fluid collection assembly 720 to be used more discretely than if the sheath 748 was not substantially flat thereby avoiding possibly embarrassing scenarios.

When the sheath 748 is substantially flat, the porous material 700 occupies substantially all of the chamber 726 and the penis receiving area 762 is collapsed (shown as being non-collapsed in FIG. 7 for illustrative purposes to show the penis receiving area 762). In other words, the sheath 748 may not define a region that is constantly unoccupied by the porous material 700. When the porous material 700 occupies substantially all of the chamber 726, the bodily fluids discharged into the chamber 726 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 758, the second panel 760, and the porous material 700 may be selected to be relatively flexible. The first panel 758, the second panel 760, and the porous material 700 are relatively flexible when the first panel 758, the second panel 760, and the porous material 700, respectively, are unable to maintain their shape when unsupported. The flexibility of the first panel 758, the second panel 760, and the porous material 700 may allow the sheath 748 to be substantially flat, as discussed above. The flexibility of the first panel 758, the second panel 760, and the porous material 700 may also allow the sheath 748 to conform to the shape of the penis even when the size and shape of the penis changes (e.g., becomes erect) and to minimize any unoccupied spaces in the chamber 726 in which bodily fluids may pool.

As previously discussed, the fluid collection assembly 720 includes a base 746 that is configured to be attached to the sheath 748. For example, the base 746 is configured to be permanently attached to the sheath 748. The base 746 is configured to be permanently attached to the sheath 748 when, for example, when the fluid collection assembly 720 is provided with the base 746 permanently attached to the sheath 748 or the base 746 is provided without being permanently attached to the sheath 748 but is configured to be permanently attached to the sheath 748 at some point in the future. Permanently attached means that the sheath 748 cannot be detached from the base 746 without damaging at least one of the sheath 748 or the base 746, using a blade to separate the sheath 748 from the base 746, and/or using chemicals to dissolve the adhesive that attaches the sheath 748 from the base 746. The base 746 may be permanently attached to the sheath 748 using an adhesive, sewing, heat sealing, RF welding, or US welding. In an embodiment, the base 746 is configured to be reversibly attached to the sheath 748. In an embodiment, the base 746 is integrally formed with the sheath 748.

The base 746 includes an aperture 750. The base 746 is permanently attached to the distal end region 740 of the sheath 748 such that the aperture 750 is aligned with the opening 724.

The base 746 is sized, shaped, and made of a material to be coupled to the skin that surrounds the penis (e.g., mons pubis, thighs, testicles, and/or perineum) and have the penis disposed therethrough. For example, the base 746 may define an aperture 750 configured to have the penis positioned therethrough. In an example, the base 746 may exhibit the general shape or contours of the skin surface that the base 746 is configured to be coupled with. The base 746 may be flexible, thereby allowing the base 746 to conform to any shape of the skin surface and mitigate the base 746 pulling the on skin surface. The base 746 may extend laterally past the sheath 748 thereby increasing the surface area of the skin of the individual to which the fluid collection assembly 720 may be attached compared to a substantially similar fluid collection assembly 720 that did not include a base.

Further examples of fluid collection assemblies that may include the porous materials disclosed herein are disclosed in U.S. patent application Ser. No. 15/612,325 filed on Jun. 2, 2017, U.S. patent application Ser. No. 15/260,103 filed on Sep. 8, 2016, U.S. Pat. No. 10,390,989 filed on Sep. 8, 2016, U.S. Provisional Patent Application No. 63/067,542 filed on Aug. 19, 2020, and U.S. patent application Ser. No. 16/433,773 filed on Jun. 6, 2019, the disclosures of each of which are incorporated herein, in its entirety, by this reference.

FIG. 8 is a block diagram of a fluid collection system 870 for fluid collection assembly 820, according to an embodiment. The fluid collection system 870 includes a fluid collection assembly 820, a fluid storage container 872, and a vacuum source 874. The fluid collection assembly 820 may be the same or substantially similar to any of the fluid collection assemblies disclosed herein. The fluid collection assembly 820, the fluid storage container 872, and the vacuum source 874 may be fluidly coupled to each other via one or more conduits 830. For example, fluid collection assembly 820 may be operably coupled to one or more of the fluid storage container 872 or the vacuum source 874 via the conduit 830. The bodily fluids collected in the fluid collection assembly 820 may be removed from the fluid collection assembly 820 via the conduit 830 which protrudes into the fluid collection assembly 820. For example, an inlet of the conduit 830 may extend into the fluid collection assembly 820, such as to a reservoir therein. The outlet of the conduit 830 may extend into the fluid collection assembly 820 or the vacuum source 874. Suction force may be introduced into the chamber of the fluid collection assembly 820 via the inlet of the conduit 830 responsive to suction (e.g., vacuum) force applied at the outlet of the conduit 830.

The suction force may be applied to the outlet of the conduit 830 by the vacuum source 874 either directly or indirectly. The suction force may be applied indirectly via the fluid storage container 872. For example, the outlet of the conduit 830 may be disposed within the fluid storage container 872 and an additional conduit 830 may extend from the fluid storage container 872 to the vacuum source 874. Accordingly, the vacuum source 874 may apply suction to the fluid collection assembly 820 via the fluid storage container 872. The suction force may be applied directly via the vacuum source 874. For example, the outlet of the conduit 830 may be disposed within the vacuum source 874. An additional conduit 830 may extend from the vacuum source 874 to a point outside of the fluid collection assembly 820, such as to the fluid storage container 872. In such examples, the vacuum source 874 may be disposed between the fluid collection assembly 820 and the fluid storage container 872.

The fluid storage container 872 is sized and shaped to retain bodily fluids therein. The fluid storage container 872 may include a bag (e.g., drainage bag), a bottle or cup (e.g., collection jar), or any other enclosed container for storing bodily fluids such as urine. In some examples, the conduit 830 may extend from the fluid collection assembly 820 and attach to the fluid storage container 872 at a first point therein. An additional conduit 830 may attach to the fluid storage container 872 at a second point thereon and may extend and attach to the vacuum source 874. Accordingly, a vacuum (e.g., suction) may be drawn through fluid collection assembly 820 via the fluid storage container 872. Bodily fluids, such as urine, may be drained from the fluid collection assembly 820 using the vacuum source 874.

The vacuum source 874 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 874 may provide a vacuum or suction to remove bodily fluids from the fluid collection assembly 820. In some examples, the vacuum source 874 may be powered by one or more of a power cord (e.g., connected to a power socket), one or more batteries, or even manual power (e.g., a hand operated vacuum pump). In some examples, the vacuum source 874 may be sized and shaped to fit outside of, on, or within the fluid collection assembly 820. For example, the vacuum source 874 may include one or more miniaturized pumps or one or more micro pumps. The vacuum sources 874 disclosed herein may include one or more of a switch, a button, a plug, a remote, or any other device suitable to activate the vacuum source 874.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable layer at least defining a chamber, at least one opening, and a fluid outlet; and
a porous material disposed in the chamber;
a conduit disposed in the chamber, the conduit defining a first passageway and a second passageway that is distinct and separate from the first passageway;
a shape memory material disposed in the second passageway, the shape memory material capable of retaining a selected shape; and
at least one plug positioned in the second passageway.

2-21. (canceled)

22. The fluid collection assembly of claim 1, wherein the porous material includes a fluid permeable inner layer and a fluid permeable outer layer.

23. The fluid collection assembly of claim 1, wherein the porous material includes a first porous layer, a second porous layer, and a supporting layer positioned and extending between at least substantially all of a length of the first porous layer and at least substantially all of a length the second porous layer.

24. The fluid collection assembly of claim 1, wherein the porous material includes a first porous layer, a second porous layer, and a supporting layer positioned and extending between the first porous layer and the second porous layer, the first porous layer including polypropylene.

25. The fluid collection assembly of claim 1, wherein the porous material includes a first porous layer, a second porous layer, and a supporting layer positioned and extending between the first porous layer and the second porous layer, the supporting layer including bamboo.

26. The fluid collection assembly of claim 1, wherein the porous material includes a first porous layer, a second porous layer, and a supporting layer positioned and extending between the first porous layer and the second porous layer, the second porous layer including polyester.

27. The fluid collection assembly of claim 1, wherein the porous material includes a first porous layer, a second porous layer, and a supporting layer positioned and extending between the first porous layer and the second porous layer, the second porous layer including a foam.

28. The fluid collection assembly of claim 1, wherein the porous material includes a first porous layer, a second porous layer, and a supporting layer positioned and extending between the first porous layer and the second porous layer, the first porous layer including polypropylene, the supporting layer including bamboo, and the second porous layer including polyester.

29. The fluid collection assembly of claim 1, wherein the conduit includes an exterior wall exhibiting a generally cylindrical shape.

30. The fluid collection assembly of claim 1, wherein the first passageway is configured to remove bodily fluids from the chamber.

31. The fluid collection assembly of claim 1, wherein the conduit includes an inner wall separating the first passageway from the second passageway.

32. The fluid collection assembly of claim 31, wherein the conduit includes an exterior wall and the inner wall exhibits single piece construction with the exterior wall.

33. The fluid collection assembly of claim 31, wherein the conduit includes a generally cylindrical exterior wall and the first passageway exhibits a generally crescent shape.

34. The fluid collection assembly of claim 1, wherein the second passageway exhibits a cross-sectional area that is smaller than a cross-sectional area of the first passageway.

35. The fluid collection assembly of claim 34, wherein the cross-sectional area of the second passageway is at least 5 times smaller than the cross-sectional area of the first passageway.

36. The fluid collection assembly of claim 1, wherein the shape memory material is capable of being shaped into the selected shape.

37. The fluid collection assembly of claim 1, wherein the shape memory material includes aluminum or an aluminum alloy.

38. The fluid collection assembly of claim 1, wherein the shape memory material includes at least one wire.

39. The fluid collection assembly of claim 38, wherein a length of the at least one wire is generally aligned with a longitudinal axis of the fluid impermeable layer.

40. The fluid collection assembly of claim 38, wherein a length of the at least one wire is about 50% to about 100% of a length of the conduit disposed in the chamber.

41. The fluid collection assembly of claim 1, wherein the plug is disposed at or near the fluid outlet.

42. The fluid collection assembly of claim 1, wherein the plug forms a substantially fluid tight seal in the second passageway.

43. A fluid collection system, comprising:
a fluid collection assembly including:
a fluid impermeable layer at least defining a chamber, at least one opening, and a fluid outlet; and
a porous material disposed in the chamber;
a conduit disposed in the chamber, the conduit defining a first passageway and a second passageway is distinct and separate from the first passageway;
a shape memory material disposed in the second passageway; and
at least one plug positioned in the second passageway;
a fluid storage container; and
a vacuum source;
wherein the chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

44. The fluid collection system of claim 43, wherein the first passageway is in fluid communication with the fluid storage container and the vacuum source.

45. The fluid collection system of claim 43, wherein the second passageway is not in fluid communication with the fluid storage container and the vacuum source.
